(19) United States
(12) Patent Application Publication
(10) Pub. No.: US 2022/0031863 A1
(43) Pub. Date: Feb. 3, 2022

(54) NANOPARTICLE DELIVERY SYSTEM

(71) Applicant: MASTER DYNAMIC LIMITED, Hong Kong (CN)

(72) Inventors: Ka Wing CHENG, Hong Kong (CN); Koon Chung HUI, Hong Kong (CN); Ching Tom KONG, Hong Kong (CN)

(73) Assignee: MASTER DYNAMIC LIMITED, Hong Kong (CN)

(21) Appl. No.: 17/299,568

(22) PCT Filed: Dec. 3, 2019
(86) PCT No.: PCT/CN2019/122687

§ 371 Date: Jun. 3, 2021

(30) Foreign Application Priority Data
Dec. 3, 2018 (HK) 18115449.5

(51) Int. Cl. A61K 47/69; A61K 47/64

(52) U.S. Cl. CPC A61K 47/6929; A61K 47/64; A61K 47/6923

(57) ABSTRACT

A conjugate (110) comprising a nanoparticle (210) for delivery of a drug to a treatment site in the body of a subject; a drug molecule (220) releasably linked to said nanoparticle (210), wherein said drug molecule (220) has a therapeutic effect at the treatment site in the body of the subject; and a disease targeting molecule (230) releasably linked to said nanoparticle (210); wherein upon said conjugate (110) being adjacent diseased tissue (240) of a subject, said disease targeting molecule (230) retains the conjugate (110) adjacent said diseased tissue (240); said drug molecule (220) is released from said nanoparticle (110) so as to provide a therapeutic effect to said diseased tissue (240); and said disease targeting molecule (230) is subsequently released from said nanoparticle (210) such that retention of the nanoparticle (210) is released, and such that the nanoparticle (210) is dispersible from said diseased tissue (240).

### TECHNICAL FIELD

The present invention relates to a nanoparticle delivery system and conjugate thereof. More particularly, the present invention provides a nanoparticle delivery system for delivery of a drug product to the body of a subject.

### BACKGROUND OF THE INVENTION

Chemotherapy is currently considered the most common method of treatment of cancers in persons throughout the world.

However, as is known, substantially that all such drugs as used in chemotherapy, as well as when utilised in other therapeutic applications, inherently come with associated side effects.

In clinical practice, side effects are still considered to be a challenging problem in cancer therapy and clinical treatment of subjects. Reduction of the side effects associated with drugs or therapy is considered an important issue, as this also encompasses the actual therapeutic effect of the drug.

Design of a drug delivery system (DDS) can be utilised to solve or at least ameliorate some of the issues associated with drug side effects, as well as addressing clinical loading and in some case solubility related issues.

A DDS can include specific drug targeting/delivery, reduced toxicity whilst maintaining requisite therapeutic effects, and the development of novel and safer medical products.

Many advanced drug delivery systems (DDS) which have been developed, having the purposes of improving the bioavailability of a drug product or active pharmaceutical ingredient (API), which may prevent or reducing premature degradation as well as enhancing drug uptake, which can be shown to maintain drug concentration within a requisite therapeutic level by controlling the drug release rate to a subject, which has been shown to reduce side effects by targeting diseased site and target cells.

Within the prior art, there have been many attempts and developments in drug delivery systems, for example targeted cancer therapy by combining new or novel materials in order to carry and deliver anti-cancer drugs, so as to minimize or reduce side effects during delivery process of the drug to a subject.

Within the prior art, various nanostructured materials have been proposed for the use in biology and medicine, for use in bio-imaging and also for use in drug delivery.

Further and within the prior art, nanoparticle-carrier type devices have been used in drug delivery for drug targeted transport and controlled release of drugs, and for the release of active pharmaceutical ingredients (APIs) and/or other therapeutic compounds. It has been shown, for example, that the nanoparticles stimulate the endocytosis of drug resistant cells so as to raise intracellular drug concentration.

However, there has been concern with the use of delivery particles, such as nanoparticles, with issues pertaining to toxicity and accumulation within the body of a subject, as well as clumping together and accumulation of such delivery particles, and thus the efficient loading of particles with drugs or therapeutic molecules or compounds, as well as effective and constant delivery to the body of a subject.

Furthermore, a drug delivery system should be able to appropriately control the release and delivery of an API or therapeutic molecules or compounds to a subject, so as to have a requisite release profile which is appropriate for the particular clinical application, for example providing a requisite blood plasma concentration or level to a subject.

Accordingly, in order to overcome such drawbacks of the prior art and associated therewith, in order to improve the quality of life of the subject and the clinical treatment thereof, improved drug delivery products, compositions and systems are required.

### OBJECT OF THE INVENTION

It is an object of the present invention to provide a nanoparticle delivery system which overcomes or at least partly ameliorates at least some deficiencies as associated with the prior art.

### SUMMARY OF THE INVENTION

In a first aspect, the present invention provides a conjugate comprising:
a nanoparticle for delivery of a drug to a treatment site in the body of a subject,
a drug molecule releasably linked to said nanoparticle, wherein said drug molecule has a therapeutic effect at the treatment site in the body of the subject; and
a disease targeting molecule releasably linked to said nanoparticle;
   wherein upon said conjugate being adjacent diseased tissue of a subject, said disease targeting molecule retains the conjugate adjacent said diseased tissue;
   said drug molecule is released from said nanoparticle so as to provide a therapeutic effect to said diseased tissue; and
   said disease targeting molecule is subsequently released from said nanoparticle such that retention of the nanoparticle is released, and such that the nanoparticle is dispersible from said diseased tissue.

Preferably, the nanoparticle is a nanodiamond which has a size in the range of from 25 nm to 80 nm, more preferably in the range of from 35 nm to 65 nm, and more preferably has a size of about 50 nm.

Preferably the disease targeting molecule is a cancer targeting molecule and wherein the diseased tissue is cancerous tissue.

Preferably, the drug molecule is an anthracycline. Preferably, the drug molecule is doxorubicin hydrochloride C₂₇H₂₉NO₁₁ (DOX).

Alternatively, the drug molecule may bemethotrexate C₂₀H₂₂N₈O₅ (MTX).

The drug molecule may be attached to the nanoparticle by intermolecular forces, and whereby the drug molecule is releasable from the nanoparticle by a change in acidic environment adjacent said diseased tissue.

Preferably, the nanoparticle is a nanodiamond, and the drug molecule is attached to a human serum albumin coating on nanodiamond by intermolecular forces, and whereby the drug molecule is releasable from the nanoparticle by a change in acidic environment adjacent said diseased tissue so as to deliver the drug molecule to the diseased tissue of the subject.

The disease targeting molecule may be linked to the nanoparticle by ethylene-vinyl acetate polymer, and wherein the linkage between the nanoparticle and the disease targeting molecule is decomposable by heat generated by irradiating the nanoparticle by infrared irradiation so as to release the nanoparticle from the diseased tissue of the subject.

Preferably the disease targeting molecule is a cancer. Preferably, the cancer targeting molecule is gastrin.

In a second aspect, the present invention provides pharmaceutical solution, comprising a plurality of a conjugates according to the first aspect and a liquid carrier.

Preferably, the solution is a solution for intravenous delivery to a subject.

In a third aspect, the present invention provides the use of a conjugate of the first aspect for the manufacture of a medicament for the prevention or treatment of a subject.

In a fourth aspect, the present invention provides method of providing therapeutic treatment to a subject in need thereof, said method including the step of delivering to the subject a therapeutic amount of the pharmaceutical solution of the second aspect.

### BRIEF DESCRIPTION OF THE DRAWINGS

In order that a more precise understanding of the above-recited invention can be obtained, a more particular description of the invention briefly described above will be described by reference to specific embodiments thereof that are illustrated in the appended drawings. The drawings presented herein may not be drawn to scale and any reference to dimensions in the drawings or the following description is specific to the embodiments disclosed.

FIG. 1 shows a schematic representation of nanoparticles of the Prior Art, clumped at a tissue site of a subject;

FIG. 2a shows a schematic representation of a conjugate according to the present invention;

FIG. 2b shows a schematic representation of the conjugate of FIG. 2a engaged with cancerous tissue;

FIG. 2c shows the conjugate of FIG. 2a and FIG. 2b with a drug molecule being released from the nanoparticle of the conjugate of FIG. 2a;

FIG. 2d shows the conjugate of FIGS. 2a to 2c with the cancer targeting molecule of FIGS. 2a to 2c being released from the nanoparticle of said conjugate; and

FIG. 2e shows the conjugate of FIGS. 2a to 2d released from the cancerous tissue of FIGS. 2a to 2d.

### DETAILED DESCRIPTION OF THE DRAWINGS

The present inventor has identified shortcomings of nanoparticle drug delivery system of the prior art, and upon identification of the problems with the prior art, has provided a system and conjugate which overcomes or at least ameliorates the problems of the prior art.

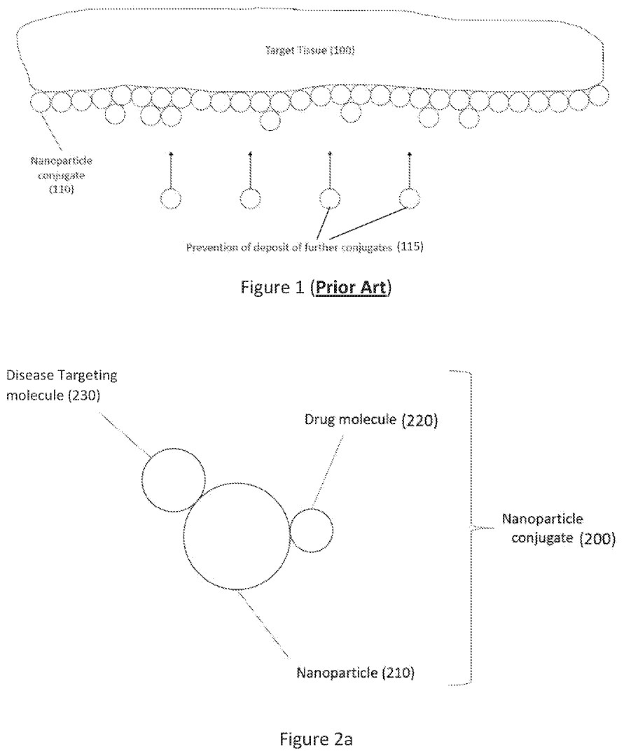

FIG. 1 shows a schematic representation of nanoparticle conjugates 110 of the Prior Art, clumped at a tissue site 100 of a subject.

Nanoparticles have been used in conjugation with disease targeting molecules to target certain tissues 100. Such tissues, such as cancer or infected tissues, have certain antigens on the cell surfaces.

The disease targeting molecules conjugated with the nanoparticles combine with the antigen such that the nanoparticle conjugate 110 can be bound to the tissue cell surface.

This effect is particularly used in bio-labelling for identifying specific tissues, such as cancer or infected tissues for example. Another usage of nanoparticles is for targeted drug delivery. Nanoparticles can conjugate with both disease targeting molecules and drug molecules for providing a therapeutic effect to the tissue of a subject.

The disease targeting molecules facilitate the binding of nanoparticle conjugates 110 to specific tissue 100 cell surfaces. After binding, the drug molecules conjugated can be released spontaneously, or under external influences, resulting in targeted drug delivery to the tissue of the subject.

However, after finishing the desired mission of delivery the drug molecule to the tissue, as is noted by the present inventors, the residual nanoparticles cause concern in the long term, and accumulative effects on the safety of the body of the subject.

For example, some nanoparticles may have certain reactivity, and resulting accumulation in the body, is considered highly undesirable. Even in the event of the usage of some stable nanoparticles, as they may form large-size aggregations while accumulated in human body, and block the passage of capillaries or renal tubules for example, and concern from a clinical standpoint thus exists.

In the case of drug delivery, the accumulated nanoparticle complexes on the tissue surface can also prevent further deposition of further nanoparticle conjugates 115, due to the relatively large sizes of the nanoparticles in comparison with antigens. Therefore, the present inventors have determined that removal of the nanoparticle conjugates 110 after completing the desired mission of drug delivery is required for at least increased or more efficient delivery of drug molecules for therapeutic effect.

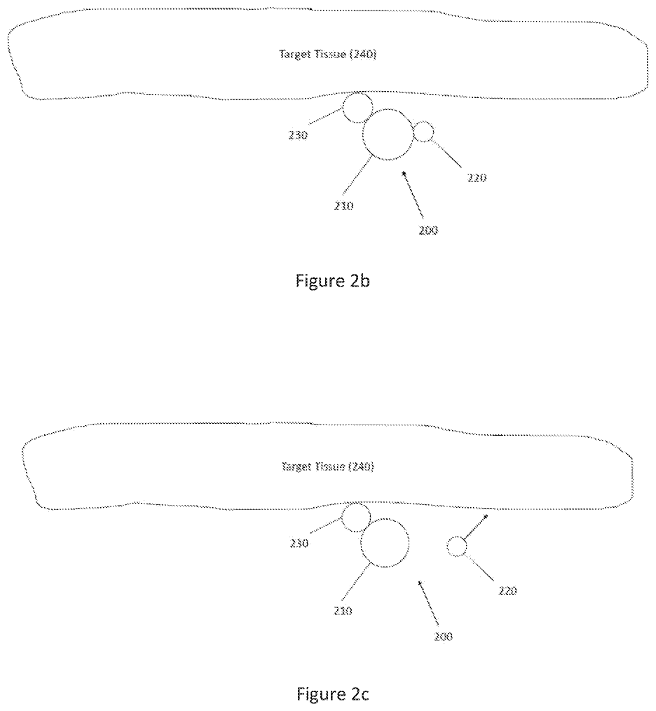

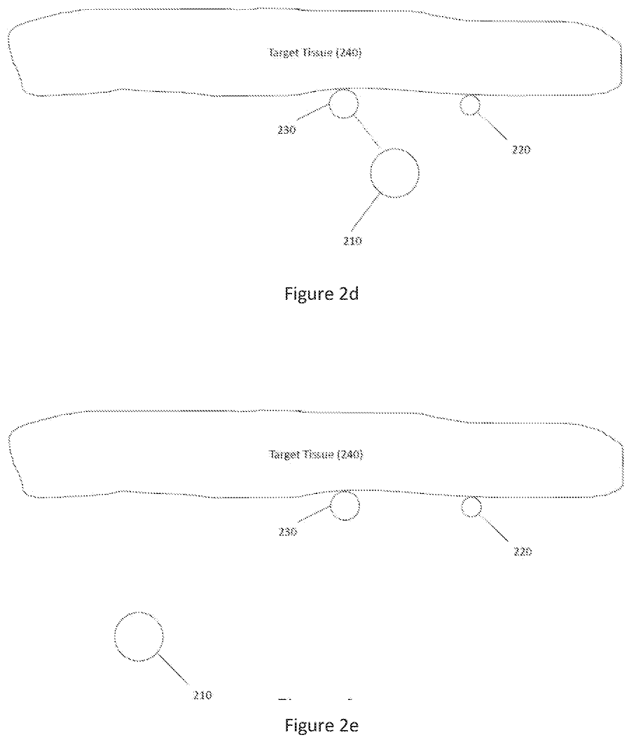

Having identified the shortcomings of the prior art, the present inventors have provided a solution as shown and described with reference Referring to FIGS. 2a to 2e as described and shown as follows.

Referring to FIG. 2a, there is shown a nanoparticle-disease targeting molecule-drug conjugate 200 according to the present invention. The nanoparticle 210 is conjugated with disease targeting molecule 230 and drug molecule 220.

As will be understood by those skilled in the art, such a figure is not to scale and merely schematically representative of a clinical environment, and further as will be understood, numerous disease targeting molecules 230 and drug molecules 220 could be provided on the nanoparticle 210, not necessarily just one which is merely from illustrative purposes.

Referring to FIG. 2b, the disease targeting molecule 230 can target diseased tissue 240, and then bind the conjugate to the tissue 240.

Referring now to FIG. 2c, after the attachment of the conjugate 200 to the tissue 240 by disease targeting molecule 230, or when the conjugate is in an appropriate proximity of the diseased tissue 240, the drug molecule 220 can be released spontaneously or under external influence from the nanoparticle 230. The drug molecule 220 released can act locally on the target diseased tissue 240, for example cancer tissue.

As shown now in FIG. 2d, after finishing the mission of delivery of the drug molecule 220 to the target diseased tissue 240, the nanoparticle 210 can be released from the disease targeting molecule 230 spontaneously or under external influence.

Thus, as is now shown in FIG. 2e, the nanoparticle 210 can move away from the tissue 240 and be removed, and thus allow deposit of further conjugates and delivery of their respective drug molecules to the tissue, by virtue of the nanoparticle being released from the tissue 240 and thus preventing the undesirable effects of blockage and localized clumping as identified by the present inventors.

As will be understood by those skilled in the art, the drug molecule 220 may be released from the nanoparticle 210 at the time of the disease targeting molecule 230 attaches to the tissue 240, after the disease targeting molecule 230 attaches to the tissue 240, or even before the disease targeting molecule 230 attaches to the tissue 240 in the case that the conjugate 200 is at a suitable proximity to the tissue 240, such that the drug molecule 220 may provide an appropriate clinical effect thereto.

Within the present invention, in a preferred embodiment, the nanoparticle 210 can be nanodiamond. Nanodiamonds are diamonds in nature so they are chemically stable. For nanodiamonds containing color centers, such NV (nitrogen vacancy) centers, these can also be used as a fluorescence agent in biolabeling, which may be useful in some applications of the invention.

The surface of nanodiamonds can be modified with different functional groups, such as —COOH, —NH₂, —OH, by way of example. These functional groups allow for nanodiamonds to react with different molecules and link together, and for a bond or adhesion effect.

Beside functional groups, sp2 carbons can also be found on nanodiamonds. These sp2 carbons are capable of absorbing infrared light so as to generate heat.

In accordance with embodiments of the present invention, nanodiamond can be conjugated with gastrin molecules, which is a growth hormone.

For applications of treatment of gastrointestinal cancers in accordance with the present invention, receptors of gastrin have been found in large abundance because of the high growing properties of cancer cells. Therefore, gastrin can be used as a cancer disease targeting molecule 230 to target cancer tissues 240, as an example of such a cancer targetter molecule.

The conjugation of nanodiamond 210 and gastrin 230 can be performed by the reaction with EDC (1-ethyl-3-(3-dimethylaminopropyl)carbodiimide hydrochloride) and sulfo-NHS (N-hydroxysulfosuccinimide).

The crosslinker reaction creates covalent bond linking —COOH and —NH₂ functional groups. The nanoparticle nanodiamond 210 and gastrin 230 can be linked together directly by this reaction, for example.

Further, a polymer may also be involved in the reaction to act as a bridge between nanodiamond 210 and gastrin 230. By way of example, such a polymer may be ethylene-vinyl acetate polymer, which engineered with —COOH or —NH₂ group to involve in the crosslinker reaction with EDC and sulfo-NHS.

The ethylene-vinyl acetate polymer breaks at high temperature so the heat generated by irradiating nanodiamond 210, which can be used to release nanodiamond 210 from the cancer targetter gastrin 230 so as to allow the nanodiamond to be displaced from the diseased tissue as described with reference to the present invention.

Doxorubicin (DOX), an anthracycline, is a chemotherapeutic drug providing a therapeutic effect acting on cancer cell tissue 240. It can be used as a drug molecule 220 to form nanoparticle-disease targeting molecule-drug conjugate 200 in accordance with the present invention.

Nanodiamonds can be conjugated with DOX by the mixing with human serum albumin (HSA). Because of the intermolecular forces, such as Van der Waal forces and hydrogen bonds, HSA can form a layer coating on the nanodiamond surface with DOX loading on it. This process is pH sensitive, and the DOX drug molecule 220 can be released in acidic environment, such as the acidic environment surrounding cancer cells in tissue, so as to provide a requisite therapeutic effect.

In an embodiment of the present invention, the whole system of conjugate can be nanodiamond 210 conjugated with cancer targetter gastrin 230 by ethylene-vinyl acetate polymer. DOX, acting as the drug molecule 220, is conjugated with nanodiamond with HSA.

The nanodiamond-gastrin-DOX conjugate can target cancer tissue 240 and release the drug DOX 220. After or at about the time of releasing the drug, infrared light can be irradiated on the cancer tissue 240 so as to release the nanodiamond 210.

The nanodiamond 210 can hence be removed without the risk of accumulating and conjugating in the body, and preventing the delivery of further DOX via further conjugates.

This nanodiamond-gastrin-DOX conjugate can be used, for example, in the treatment of gastrointestinal cancers. The released DOX acts on cancer tissue in the canal and then the nanodiamonds are released and removed by excretion.

In other embodiments, similar applications can also be done on skin of a subject. The target tissue is not necessarily to be cancer tissue, such as melanoma. Any skin abnormality, such as wounds or pigmentation, which can also be targeted with a disease targeting molecule, can also be treated with nanodiamond-targetter-drug conjugates, and delivery of a therapeutic agent thereto.

As will be understood by those skilled in the art, the above embodiments of the invention are exemplary embodiments, and whilst the examples generally pertain to the use of nanodiamonds as the nanoparticle, and for the treatment of cancer, in other and alternate embodiments, the nanoparticles may be other nanoparticles and the tissue disorder may be other disorders or diseases other than cancer.

The present invention has been described, in the experimental embodiments, using doxorubicin hydrochloride, C₂₇H₂₉NO₁₁, known as DOX. DOX is chemotherapy medication used to treat cancer. This includes breast cancer, bladder cancer, Kaposi's sarcoma, lymphoma, and acute lymphocytic leukemia. It is often used together with another chemotherapy agent, and the present invention is applicable to combination therapy.

As will be understood, other anti-cancer drugs may be used, such as Methotrexate Empirical formula: C₂₀H₂₂N₈O₅, known as MTX. Methotrexate is a chemotherapy agent and immune system suppressant. It is used to treat cancer, autoimmune diseases, ectopic pregnancy, and for medical abortions. Types of cancers it is used for includes breast cancer, leukemia, lung cancer, lymphoma, and osteosarcoma. Types of autoimmune diseases it is used for includes psoriasis, rheumatoid arthritis, and Crohn's disease. It can be given by mouth or by injection.

As will also be understood, the conjugate of the present invention may, in alternate embodiments, have more than one type of drug molecule attached thereto.

1. A conjugate comprising:
a nanoparticle for delivery of a drug to a treatment site in the body of a subject;
a drug molecule releasably linked to said nanoparticle, wherein said drug molecule has a therapeutic effect at the treatment site in the body of the subject; and
a disease targeting molecule releasably linked to said nanoparticle;
wherein upon said conjugate being adjacent diseased tissue of a subject, said disease targeting molecule retains the conjugate adjacent said diseased tissue;
said drug molecule is released from said nanoparticle so as to provide a therapeutic effect to said diseased tissue; and
said disease targeting molecule is subsequently released from said nanoparticle such that retention of the nanoparticle is released, and such that the nanoparticle is dispersible from said diseased tissue.

2. A conjugate according to claim 1, wherein the nanoparticle is a nanodiamond which has a size in the range of from 25 nm to 80 nm.

3. A conjugate according to claim 1, wherein the nanoparticle is a nanodiamond which has a size in the range of from 35 nm to 65 nm.

4. A conjugate according to claim 1, wherein the nanoparticle is a nanodiamond which has a size of about 50 nm.

5. A conjugate according to claim 1, wherein the disease targeting molecule is a cancer targeting molecule and wherein the diseased tissue is cancerous tissue.

6. A conjugate according to claim 1, wherein the drug molecule is an anthracycline.

7. A conjugate according to claim 1, wherein the drug molecule is doxorubicin hydrochloride C27H29NO11 (DOX).

8. A conjugate according to claim 1, wherein the drug molecule is Methotrexate C20H22N8O5 (MTX).

9. A conjugate according to claim 1, wherein the drug molecule is attached to the nanoparticle by intermolecular forces, and whereby the drug molecule is releasable from the nanoparticle by a change in acidic environment adjacent said diseased tissue.

10. A conjugate according to claim 1, wherein the nanoparticle is a nanodiamond, and the drug molecule is attached to a human serum albumin coating on nanodiamond by intermolecular forces, and whereby the drug molecule is releasable from the nanoparticle by a change in acidic environment adjacent said diseased tissue so as to deliver the drug molecule to the diseased tissue of the subject.

11. A conjugate according to claim 1, wherein the disease targeting molecule is linked to the nanoparticle by ethylene-vinyl acetate polymer, and wherein the linkage between the nanoparticle and the disease targeting molecule is decomposable by heat generated by irradiating the nanoparticle by infrared irradiation so as to release the nanoparticle from the diseased tissue of the subject.

12. A conjugate according to claim 11, wherein the disease targeting molecule is a cancer targeting molecule.

13. A conjugate according to claim 12, wherein the cancer targeting molecule is gastrin.

14. A pharmaceutical solution, comprising a plurality of a conjugates and a liquid carrier, wherein said conjugates comprise:
a nanoparticle for delivery of a drug to a treatment site in the body of a subject;
a drug molecule releasably linked to said nanoparticle, wherein said drug molecule has a therapeutic effect at the treatment site in the body of the subject; and
a disease targeting molecule releasably linked to said nanoparticle;
wherein upon said conjugate being adjacent diseased tissue of a subject, said disease targeting molecule retains the conjugate adjacent said diseased tissue;
said drug molecule is released from said nanoparticle so as to provide a therapeutic effect to said diseased tissue; and
said disease targeting molecule is subsequently released from said nanoparticle such that retention of the nanoparticle is released, and such that the nanoparticle is dispersible from said diseased tissue.

15. A pharmaceutical solution according to claim 14, wherein said solution is a solution for intravenous delivery to a subject.

16. The use of a conjugate for the manufacture of a medicament for the prevention or treatment of a subject, wherein said conjugate comprises:
a nanoparticle for delivery of a drug to a treatment site in the body of a subject;
a drug molecule releasably linked to said nanoparticle, wherein said drug molecule has a therapeutic effect at the treatment site in the body of the subject; and
a disease targeting molecule releasably linked to said nanoparticle;
wherein upon said conjugate being adjacent diseased tissue of a subject, said disease targeting molecule retains the conjugate adjacent said diseased tissue;
said drug molecule is released from said nanoparticle so as to provide a therapeutic effect to said diseased tissue; and
said disease targeting molecule is subsequently released from said nanoparticle such that retention of the nanoparticle is released, and such that the nanoparticle is dispersible from said diseased tissue.

17. A method of providing therapeutic treatment to a subject in need thereof, said method including the step of delivering to the subject a therapeutic amount of the pharmaceutical solution comprising a plurality of a conjugates and a liquid carrier, and wherein said conjugates comprise:
a nanoparticle for delivery of a drug to a treatment site in the body of a subject;
a drug molecule releasably linked to said nanoparticle, wherein said drug molecule has a therapeutic effect at the treatment site in the body of the subject; and
a disease targeting molecule releasably linked to said nanoparticle;
wherein upon said conjugate being adjacent diseased tissue of a subject, said disease targeting molecule retains the conjugate adjacent said diseased tissue;
said drug molecule is released from said nanoparticle so as to provide a therapeutic effect to said diseased tissue; and
said disease targeting molecule is subsequently released from said nanoparticle such that retention of the nanoparticle is released, and such that the nanoparticle is dispersible from said diseased tissue.
